(19) United States
(12) Patent Application Publication
(10) Pub. No.: US 2005/0266058 A1
(43) Pub. Date: Dec. 1, 2005

(54) Topical preparations containing ambroxol

(75) Inventors: Anke Esperester, Mainz (DE); Frieder Ulrich Maerz, Soergenloch (DE); Claudia Mueller, Appenheim (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim International GmbH, Ingelheim (DE)

(21) Appl. No.: 11/120,450

(22) Filed: May 3, 2005

(30) Foreign Application Priority Data
May 3, 2004 (DE) 10 2004 021 992

(51) Int. Cl. A61K 31/137; A61L 15/16; A61F 13/02

(52) U.S. Cl. 424/448; 514/649

(57) ABSTRACT

The present invention relates to topical pharmaceutical compositions containing ambroxol or one of the pharmacologically acceptable salts thereof, preferably in the form of the hydrochloride, for direct application or administration to the skin and/or mucosa with anti-inflammatory and local anesthetic properties.

The present invention relates to topical pharmaceutical compositions containing ambroxol or one of the pharmacologically acceptable salts thereof, preferably in the form of the hydrochloride, for direct application or administration to the skin and/or mucosa with anti-inflammatory and local anesthetic properties.

In pharmaceuticals, various base formulations for applying pharmaceutical preparations to the skin and mucosa are known. A summary is found in standard textbooks of pharmaceutical technology, e.g., U Schöffing, “Arzneiformenlehre”, Deutscher Apotheker Verlag Stuttgart, 4th Edition 2003 or Gurny/Junginger, “Bioadhesion—Possibilities and Future Trends”, Wissenschaftliche Verlagsgesellschaft Stuttgart, 1990. WO 00/38653 describes an improved formulation for the transdermal application of corticosteroids, in which ambroxol is mentioned, alongside many other antioxidants, as a suitable excipient for protecting from oxidative spoiling. Suckable ambroxol tablets are known for treating pain in the throat and pharyngeal cavity (EP 1200070, WO 03/072094). Pharmaceutically effective formulations containing ambroxol or the salts thereof as active substance for direct local application to and treatment of the skin or mucosa have not, however, been described in the prior art.

Topical formulations of compounds with an anesthetic or anti-inflammatory activity often exhibit side effects.

The aim of the present invention is therefore to provide topical formulations which, in addition to having a good anti-inflammatory and anesthetic activity, have no or only minimal side effects.

### DESCRIPTION OF THE INVENTION

It has surprisingly been found that topical pharmaceutical compositions containing ambroxol or one of the pharmacologically acceptable salts thereof for direct application to the skin and/or mucosa have anti-inflammatory and local anesthetic properties.

The exceptional toxicologically profile of ambroxol also allows such formulations to be applied to large areas and over long periods.

The present invention relates to topical pharmaceutical compositions containing ambroxol or one of the pharmacologically acceptable salts thereof for direct application or administration to the skin and/or mucosa, preferably to the skin or oral mucosa, particularly the skin, with anti-inflammatory and local anesthetic properties.

Topical pharmaceutical compositions in which ambroxol is in the form of its hydrochloride are preferred.

Also preferred are topical pharmaceutical compositions in the form of a formulation selected from among gels, hydrophilic pastes, lotions and solutions, preferably gels and hydrophilic pastes.

Particularly preferred are topical pharmaceutical compositions in the form of a formulation selected from among gels, hydrophilic pastes, lotions and solutions, wherein the content of ambroxol is from 0.1% to 20% (w/w), preferably from 0.5% to 5% (w/w).

Particularly preferred are topical pharmaceutical compositions in the form of a formulation selected from among suppositories, hydrophobic pastes, ointments, creams, lotions and sticks, preferably suppositories, hydrophobic pastes and sticks.

A preferred embodiment of the invention consists of topical pharmaceutical compositions in the form of muco-adhesive plasters, buccal strips or muco-adhesive tablets, preferably muco-adhesive plasters or buccal strips.

Another preferred embodiment of the invention consists of topical compositions, while the content of ambroxol in muco-adhesive plasters is from 1% to 50% (w/w) based on the total mass of the hydrophilic support layer, preferably 5% to 40% (w/w), most preferably from 10 to 30% (w/w).

Particularly preferred are the topical compositions described above, wherein the retention time of the ambroxol or of a pharmaceutically acceptable salt thereof on the skin and/or mucosa is longer than that of a non-ionic hydrophilic cream containing 0.1% ambroxol, according to the 2003 edition of the German Pharmacopoeia.

The present invention further relates to the use of ambroxol or one of the pharmacologically acceptable salts thereof for preparing a pharmaceutical composition for the topical treatment of pain, burning or irritation of the skin and/or mucosa, preferably pain and burning of the mucosa or irritation and burning of the skin, most preferably pain and burning of the mucosa.

The present invention also relates to the use of ambroxol or one of the pharmacologically acceptable salts thereof for preparing a pharmaceutical composition for the topical treatment of inflammation.

The present invention further relates to the use of ambroxol or one of the pharmacologically acceptable salts thereof for preparing a pharmaceutical composition for the topical treatment of conditions selected from among painful inflammation in the mouth or in the vaginal area, mosquito bites, skin rashes of allergic, immunological or idiopathic origin and itching or burning hemorrhoids, preferably painful inflammation of the mouth or vaginal area and itching or burning hemorrhoids.

Acids suitable for forming salts of ambroxol include for example hydrochloric acid, hydrobromic acid, sulphuric acid, phosphoric acid, nitric acid, oxalic acid, malonic acid, fumaric acid, maleic acid, tartaric acid, citric acid, ascorbic acid and methane sulphonic acid, preferably hydrochloric acid.

Gels, Hydrophilic Pastes, Lotions, and Solutions

The gels, hydrophilic pastes, lotions and solutions according to the invention contain different amounts of water, one or more excipients selected from among natural, semi-synthetic or synthetic polymers, inorganic gel-forming compounds, flavorings, perfumes, sweeteners, colorings, preservatives, lower alcohols, polyols, pH regulators, permeation promoters and solubilizers.

Suitable polymers are pharmaceutically acceptable compounds selected from the group comprising gum arabic, cellulose, cellulose derivatives, preferably non-ionic and mucoadhesive cellulose derivatives, particularly preferably methylcellulose (MC), carboxymethylcellulose (CMC) or the salts thereof, hydroxypropylcellulose (HPC), hydroxyethylcellulose (HEC), hydroxypropylmethylcellulose (HPMC) or methylethyl-cellulose (MEC), Polyvinylalkylether-co-maleic anhydride or the salts thereof, gelatine, pectin, polyethyleneglycols (PEG), polyvinylalcohol (PVA), polyvinylpyrrolidone (PVP), tragacanth, carrageenin, xanthan, chitosan, chitosan chloride, agarose, agar-agar, alginates, poloxamers, starch, starch derivatives, guar gum, galactomannane, polyacrylates, cross-linked acrylic polymers, poly(hydroxyethyl), poly(hydroxypropyl)- and poly(hydroxypropylmethyl)methacrylates.

Suitable inorganic gels are colloidal silicon dioxides or bentonite.

The term lower alcohols in the present invention denotes ethanol, 1-propanol and 2-propanol.

Suitable polyols are compounds selected from among ethyleneglycol, propyleneglycol, glycerol and sugar alcohols, preferably glycerol, sorbitol, and maltitol.

Compounds which are suitable as pH regulators and permeation promoters correspond to the excipients listed in the section on hydrophilic ointments, pastes, creams, and lotions. Solubilizers, perfumes, colorings, sweeteners, and preservatives may be added in pharmaceutically acceptable amounts.

To prepare the hydrophilic pastes or lotions described above, finely ground insoluble inorganic compounds, for example zinc oxide and titanium dioxide may be added.

The mucoadhesive plasters according to the invention consist of at least one hydrophilic layer and optionally a more hydrophobic covering layer, which is optionally linked to the mucoadhesive layer via a separate connecting layer. The hydrophilic layer contains ambroxol or one of the pharmaceutically acceptable salts thereof, for example in a concentration ranging from 1% to 50% (w/w), preferably from 5% to 40% (w/w), particularly preferably from 10% to 30% (w/w) ambroxol, based on the total mass of the dried hydrophilic layer.

The hydrophilic mucoadhesive layer contains one or more natural, semisynthetic or synthetic hydrocolloidal polymers and optionally one or more plasticizers. Moreover, pharmaceutically acceptable excipients, for example excipients which influence adhesive qualities and/or flexibility, crystallization inhibitors, flavorings, perfumes, sweeteners, colorings, preservatives, lower alcohols, permeation enhancers, pH-regulators, and/or solubilizers may be present.

The covering layer contains a natural, semisynthetic or synthetic film-forming compound, which is insoluble or poorly soluble in water and has inferior mucoadhesive properties to those of the hydrocolloidal polymer in the hydrophilic layer, preferably selected from among the polyacrylates and cellulose derivatives. Preferably, the covering layer also contains one or more plasticizers and, optionally, flavorings, perfumes, sweeteners, and colorings.

In order to prepare the covering layer, the film-forming component may be used in the form of an aqueous dispersion, which contains other additives for stabilizing the dispersion and/or assisting with film formation, for example surfactants, preservatives or antifoamers.

The covering layer may be prepared separately and may contain plastic materials suitable for pharmaceutical use, for example polyethylene, polyethylene terephthalate, polypropylene, and/or polyvinyl chloride.

The mucoadhesive plaster may also contain a connecting layer for securing the functional layers. The connecting layer comprises a polymer with suitable adhesive qualities and optionally plasticizers, colorings, and other excipients which influence adhesive qualities and/or flexibility.

Suitable hydrocolloidal polymers include compounds selected from among the mucoadhesive cellulose derivatives, for example methylcellulose (MC), carboxymethyl-cellulose (CMC), hydroxypropylcellulose (HPC), hydroxyethylcellulose (HEC), hydroxypropylmethylcellulose (HPMC), methylethylcellulose (MEC), gelatine, soluble starch and the pharmacologically acceptable derivatives thereof, pectin, tragacanth, alginic acid and the pharmacologically acceptable salts thereof, guar gum, karaya gum, poly(ethylene oxide), polyvinylalcohol (PVA), polyvinylpyrrolidone (PVP), polyvinylacetate, polyvinylalkylether-co-maleic anhydride and the pharmacologically acceptable salts thereof, polyacrylates, cross-linked acrylic polymers, poly(hydroxyethyl)methacrylate, poly(hydroxypropyl) methacrylate, poly(hydroxypropylmethyl)methacrylate and mixtures of these compounds with polyisobutylene.

The film-forming compounds used may be regenerated cellulose (cellophane), hydrophobic cellulose derivatives, for example hydroxypropylcellulose (HPC), ethylcellulose (EC) or celluloseacetate, polyacrylate, polymethacrylate, poly(hydroxyethyl)methacrylate, poly(hydroxypropyl)methacrylate or poly(hydroxypropylmethyl)methacrylate.

Plasticisers that may be used are phthalates, for example, dibutylphthalate, sebacate, e.g., dibutylsebacate, adipates, for example, dibutyladipate, polyols, for example, alkylene-glycols, glycerol or polyethyleneglycol, sugar alcohols, for example, sorbitolol or maltitol, triacetin, or triethylcitrate.

The polymeric binders may consist of agarose, polyvinylpyrrolidone, polyvinylalcohol, polyacrylate, polymethacrylate, poly(hydroxyethyl)methacrylate, poly(hydroxypropyl) methacrylate or poly(hydroxypropylmethyl)methacrylate, as well as cellulose derivatives, for example methylcellulose (MC), carboxymethylcellulose (CMC) or hydroxypropyl-methylcellulose (HPMC).

Suitable pH-regulators and permeation promoters are compounds as described under hydrophilic ointments, pastes, creams and lotions.

The solubilizers used according to the invention, flavorings, colorings, sweeteners and preservatives are pharmaceutically acceptable excipients.

Mucoadhesive Tablets

The mucoadhesive tablets according to the invention contain ambroxol or one of the pharmaceutically acceptable salts thereof in a concentration of 0.1% to 30% (w/w), preferably 1% to 20% (w/w) ambroxol. They also contain at least one mucoadhesive polymer and optionally other excipients, for example binders, fillers, flow agents, and lubricants. They may optionally contain pH-regulators and/or permeation promoters. In addition, perfumes, flavorings, sweeteners, and/or colorings may be added.

Suitable mucoadhesive polymers according to the invention are cellulose or their derivatives, preferably non-ionic cellulose derivatives, for example, methylcellulose (MC), carboxymethylcellulose (CMC), hydroxypropylcellulose (HPC), hydroxyethylcellulose (HEC), hydroxypropylmethylcellulose (HPMC) or methylethylcellulose (MEC), polyvinylalkylether-co-maleic anhydride) or the salts thereof, gelatine, pectin, polyethyleneglycol (PEG), polyvinylalcohol (PVA), polyvinylpyrrolidone (PVP), polyvinylacetate, tragacanth, carrageenin, xanthan, chitosan, chitosan chloride, agarose, agar-agar, alginic acid or the salts thereof, poloxamers, starch, starch derivatives, guar gum, galactomannan, polyacrylate, polymethacrylate, poly(hydroxyethyl)methacrylate, poly(hydroxypropyl)methacrylate or poly(hydroxypropylmethyl)methacrylate.

The binders and fillers used are pharmaceutically acceptable excipients, for example, starch or starch derivatives, cellulose or the derivatives thereof, dextrin, tragacanth, gelatine, polyvinylpyrrolidone, polyvinylalcohol, sugars such as sucrose or lactose, sugar alcohols, or calcium phosphates.

Flow agents and lubricants are preferably selected from pharmaceutically acceptable compounds from the group comprising talc, colloidal silica, stearic acid or the salts thereof, fats, for example, glyceryltribehenate, waxes, polyethyleneglycols, and fumaric acid.

The flavorings, colorings, and sweeteners used according to the invention are pharmaceutically acceptable excipients.

Suitable pH-regulators and permeation promoters are the compounds described below under hydrophilic ointments, pastes, creams, and lotions.

Hydrophobic Ointments, Pastes, and Suppositories

The ointments, pastes, and suppositories according to the invention consist of a lipophilic base in which ambroxol or one of the pharmaceutically acceptable salts thereof is dissolved or dispersed. They may additionally contain pharmaceutically acceptable hydrocolloids to improve mucoadhesion and/or prevent recrystallisation. They may also contain pharmaceutically acceptable perfumes, sweeteners, colorings, permeation promoters, as well as preservatives and/or antioxidants.

The lipophilic base is selected from among the synthetic or natural hydrocarbons, for example, paraffins, polyethylenes or Vaseline gels, plant or animal oils or fats, hardened fats, synthetic glycerides, waxes and liquid polyalkylsiloxanes.

The pharmaceutically acceptable hydrocolloids are selected from among cellulose and its derivatives, preferably non-ionic and mucoadhesive derivatives, for example methylcellulose (MC), carboxymethylcellulose (CMC), hydroxypropylcellulose (HPC), hydroxyethylcellulose (HEC), hydroxypropyl-methylcellulose (HPMC) and methylethylcellulose (MEC), from poly (alkyl vinyl ether co-maleic anhydride) as well as the salts thereof, gelatine, pectin, poly(ethylene oxide), polyvinyl alcohol (PVA), polyvinylpyrrolidone (PVP), tragacanth, carrageenin, xanthan, chitosan, chitosan chloride, agarose, agar-agar, alginic acid and the salts thereof, poloxamer, starch, starch derivatives, guar gum, karaya gum, galactomannan, polyacrylate, polymethacrylate, poly(hydroxyethyl)methacrylate, poly(hydroxypropyl)methacrylate and poly(hydroxypropyl-methyl)methacrylate.

Suitable preservatives, antioxidants and permeation promoters are the ones listed under the following hydrophilic ointments, pastes, creams, and lotions.

Hydrophilic Ointments, Pastes, Creams, and Lotions

The hydrophilic ointments, pastes, creams, and lotions according to the invention consist of a lipophilic base and surfactant substances of the O/W and/or W/O emulsifier type. In addition, water may be present in various amounts, as desired. Depending on the amount of water and the type of emulsifier the system may be in the form of an OIW or W/O-type emulsion. Products according to this invention contain ambroxol or the salts thereof in a concentration of between 0.1% and 50%, preferably between 1% and 40%, particularly preferably in the range from 1.5%-5% in aqueous systems and 5%-30% in anhydrous systems. In addition to ambroxol and the pharmaceutically acceptable salts thereof, preservatives, antioxidants, permeation promoters, polyols, spreading agents, thickeners, colorings, flavorings as well as perfumes and pH regulators may also be incorporated.

The following pharmaceutically acceptable excipients or selected mixtures thereof are suitable as the lipophilic base:
hydrocarbons, for example white Vaseline, yellow Vaseline, thin and thick liquid paraffin, hard paraffin, microcrystalline paraffin, paraffin oil, polyethylene, squalene, or perhydrosqualene,
glycerides, for example, partial glycerides, polyglycerides, mono-, di- or triglycerides,
fatty acids, for example stearic acid, palmitic acid, or oleic acid,
fatty oils of plant origin, for example borage seeds, thistles, groundnut, coconut or maize seed oil, fatty oils of (semi)synthetic origin such as medium-chain triglycerides,
fats and hardened glycerides of plant origin, for example hardened groundnut oil, castor oil or cocoa butter,
fats of animal origin, for example pork lard, or fats of semisynthetic origin such as hard fat or shea butter,
waxes of natural and synthetic origin, for example yellow wax, bleached wax, microcrystalline wax, beeswax, cetylpalmitate or the derivatives thereof, preferably acetylated wax, polyethylene wax, cetylester wax or THG wax,
resins, for example colophony, or
silicones, for example silicone oil, dimethicone, simethicone or cyclomethicone.

The following pharmaceutically acceptable excipients may be used as surfactant substances:
anionically active emulsifiers, for example alkali metal stearate, preferably potassium stearate or metal stearate, preferably aluminium monostearate, amine soaps, preferably triethanolamine or triethanolaminelaurylsulphate, as well as alkylsulphates, preferably sodium dodecylsulphate,
cationically active emulsifiers, for example quaternary ammonium compounds, preferably benzalkonium chloride or cetylpyridinium chloride,
amphoteric emulsifiers, for example natural or synthetic phospholipids, particularly lecithin, phosphatidylcholine, phosphatidylethanolamine, phosphatidylglyceride, phosphatidylinositol, phosphatidylserine or sphingomyelins or betaine
non-ionic emulsifiers, for example higher fatty alcohols, preferably cetylalcohol, stearylalcohol or cetylstearylalcohol, partial esters of polyhydric alcohols, preferably ethylene-/propyleneglycol fatty acid ester, particularly preferably ethyleneglycol monostearate, distearate or propyleneglycol monostearate, glycerol fatty acid esters, preferably glycerol monopalmitate, glycerol dipalmitate, glycerol tripalmitate, glycerol monostearate, glycerol monoisostearate, glycerol distearate, glycerol diisostearate, glycerol tristearate, glycerol trihydroxystearate, glycerol monooleate or glycerol dioleate, sorbitolan fatty acid esters, preferably sorbitolan laurate, sorbitol palmitate sorbitol stearate, sorbitolan monooleate, sorbitolan sesquioleate, or sorbitolan trioleate, ethers and esters of polyethyleneglycol, preferably polyethyleneglycol fatty alcohol ethers, preferably polyethyleneglycol laurylether, polyethyleneglycol cetylether, polyethyleneglycol stearylether, polyethyleneglycol cetylstearylether, or polyethyleneglycol myristylcetylstearylether, polyethyleneglycol fatty acid esters, preferably polyethyleneglycol monolaurate, polyethyleneglycol monostearate, polyethyleneglycol distearate, polyethyleneglycol stearylstearate or polyethyleneglycol ricinooleate, polyethyleneglycol sorbitan fatty acid esters, preferably polysorbate, polyethyleneglycol glycerol fatty acid esters, preferably polyethyleneglycol glycerolmonostearate, polyethyleneglycol glyceroldistearate, polyethyleneglycol glycerolhydroxystearate, polyethyleneglycol glyceroltripalmitate, polyethyleneglycol glyceroltrilinolate, polyethyleneglycol glyceroltrioleate, polyethyleneglycol glycerolricinoleate or polyethyleneglycol glycerolcoccoate, stearic alcohols, preferably cholesterol or wool alcohol, block copolymers of polyoxyethylene/polyoxypropylene, preferably poloxamers, wool fat or wool alcohols as well as mixtures of two or more of the above-mentioned emulsifiers.

Suitable preservatives according to the invention are:
alcohols and phenols such as ethanol, isopropanol, benzylalcohol, chlorobutanol, phenylethylalcohol, phenoxyethanol, phenol, chlorocresol, thymol or triclosan,
carboxylic acids and the salts thereof such as benzoic acid, sodium benzoate, sorbic acid, potassium sorbate, PHB esters (4-hydroxy benzoic acid esters) preferably methyl-4-hydroxybenzoate, ethyl-4-hydroxybenzoate, propyl-4-hydroxybenzoate or butyl-4-hydroxybenzoate and the sodium compounds thereof,
nitrogen compounds such as benzalkonium chloride, chlorhexidine gluconate, pyrithione zinc or cis1-(3-chlorallyl-3,5,7 triaza-1-azonia-adamatane chloride, or
propylene carbonate
   and mixtures of two or more of the above-mentioned preservatives.

Suitable antioxidants according to the invention are natural antioxidants such as ascorbic acid, salicylic acid or α-tocopherol, semisynthetic antioxidants such as ascorbic acid or gallic acid esters, particularly palmitoylascorbic acid or propylgallate, synthetic antioxidants such as butylhydroxyanisol, butylhydroxytoluene or sulphite, particularly sodium bisulphite, complexing agents such as editic acid or sodium-EDTA, as well as mixtures of two or more of the abovementioned antioxidants.

Suitable polyols according to the invention are glycerol, sugar alcohols such as sorbitol, mannitol, maltitol or isomalt, ethyleneglycol, propyleneglycol, hexyleneglycol or polyethyleneglycols.

Suitable spreading agents according to the invention are myristylmyristate, isopropylmyristate, isopropylpalmitate, isopropyllanoate, diisopropyladipate and dibutyladipate.

Suitable pH regulators according to the invention are acids such as acetic acid, tartaric acid, citric acid, lactic acid, hydrochloric acid, sulphuric acid or phosphoric acid, bases such as ammonia, sodium hydroxide, potassium hydroxide, lithium hydroxide, aluminium hydroxide or trometamol as well as salts such as sodium hydrogen carbonate, sodium monohydrogen phosphate, sodium dihydrogen phosphate, potassium monohydrogen phosphate, potassium dihydrogen phosphate, sodium chloride, sodium citrate, sodium oxalate, sodium lactate, calcium lactate, magnesium sulphate, ammonium monohydrogen citrate or diammonium hydrogen citrate.

Suitable permeation promoters according to the invention are urea, dimethylsulphoxide, hyaluronic acid sodium salt, alkanols such as laurylalcohol or oleylalcohol, alkanoic acids such as oleic acid, 1-dodecylazacycloheptan-2-one, ethyleneglycol, propyleneglycol or menthol, as well as other permeation promoters selected from among the 1-acylglycosides, 1-acyl-polyoxyethylenes, 1-acyl-saccharides, 2-n-acyl-cyclohexanones, 2-n-acyl-1,3-dioxolanes (SEPA), 1,2,3-triacyl-glycerols, 1-alkanols, 1-alkanoic acids, 1-alkyl-acetates, 1-alkyl-amines, 1-alkyl-n-alkyl-polyoxyethylenes, 1-alkyl-alkylates, n-alkyl-beta-D-thioglycosides, 1-alkyl-glycerides, 1-alkyl-propyleneglycols, 1-alkyl-polyoxyethylenes, 1-alkyl-2-pyrrolidones, alkyl-acetoacetates, alkyleneglycols, alkylmethylsulphoxides, alkyl-propionates, alkylsulphates, diacylsuccinates, diacyl-N,N-dimethylaminoacetates (DDAA), diacyl-N,N-dimethylaminoisopropionates (DDAIP) and phenylalkylamines.

The thickeners used may be natural or semisynthetic polymers, synthetic polymers, inorganic gel-forming compounds as mentioned above in the description of the gels and hydrophilic pastes.

The flavorings, colorings, and perfumes used according to the invention are pharmaceutically acceptable excipients.

Sticks

Sticks within the scope of this invention contain 0.1% to 50% (w/w), preferably 1% to 45% (w/w) and particularly preferably 2% to 40% (w/w) ambroxol or the pharmaceutically useful salts thereof. They also contain 4% to 8% (w/w) of sodium soaps, particularly sodium soaps of palmitic acid, stearic acid, stearic acid amides and stearic acid monoethanolamines as well as ethanol, isopropanol and/or water in variable amounts by weight. Alternatively the active substance may also be incorporated in a base consisting of one or more polyethyleneglycols of different chain lengths in the form of a stick.

Moreover, emulsifiers, preservatives, antioxidants, spreading agents, polyols, permeation promoters and perfumes may be present. Excipients as described hereinbefore under “Hydrophilic ointments, pastes, creams, and lotions” may be selected from the groups specified.

The preparation of the formulations according to the invention may be carried out according to methods known from the literature.

The formulations according to the invention will be illustrated by the Examples that follow. The Examples serve as an illustration and are not intended in a restrictive capacity.

### EXAMPLES

### Example 1

| Solution (1% ambroxol HCl) | [g/100 g] |
|---|---|
| ambroxol HCl | 1.0 |
| glycerol 85% | 20.0 |
| ethanol 96% | 5.0 |
| Menthol | 0.01 |
| peppermint flavouring | 0.02 |
| indigocarmine 85% | 0.000015 |
| (colouring) | |
| purified water | 73.97 |

### Example 2

| Gel (3% ambroxol HCl) | [g/100 g] |
|---|---|
| ambroxol HCl | 3.0 |
| polyvinylpyrrolidone, | 30.0 |
| type 90 | |
| purified water | 67 |

### Example 3

| Gel (0.5% ambroxol HCl) | [g/100 g] |
|---|---|
| ambroxol HCl | 0.5 |
| polyethyleneglycol 400 | 49.75 |
| polyethyleneglycol 1000 | 49.75 |

### Example 4

| Gel (2% ambroxol HCl) | [g/100 g] |
|---|---|
| ambroxol HCl | 2.0 |
| tartaric acid | 0.1 |
| benzalkonium chloride | 0.01 |
| hydroxyethylcellulose | 5.0 |
| saccharin sodium | 0.03 |
| glycerol 85% | 10 |
| perfume (Coolfresh Aroma, | 0.24 |
| Messrs Düllberg, Hamburg) | |
| Patent blue V (colouring, | 0.003 |
| obtainable from Messrs | |
| Givaudan Deutschland GmbH, | |
| Dortmund) | |
| polysorbate 80 | 1.5 |
| purified water | ad 100.0 |

The non-swelling ingredients are dissolved in water. The gel-forming components are added and allowed to swell. The mixture is stirred gently to form a homogeneous solution or a homogeneous gel.

### Example 5

| Mucoadhesive plaster | | [%] of the |
|---|---|---|
| three-layer laminate | | layer dry mass |
| hydrocolloid polymer layer | ambroxol HCl | 20 |
| | hydroxypropylmethyl- | 72 |
| | cellulose | |
| | propyleneglycol | 8 |
| covering layer | ethylcellulose (type N14) | 90 |
| | propyleneglycol | 10 |
| binding layer | polyvinylpyrrolidone, type | 100 |
| | 90 | |

### Example 6

| Mucoadhesive plaster | | [%] of |
|---|---|---|
| two-layer laminate | | the layer dry mass |
| hydrocolloid polymer layer | ambroxol HCl | 20 |
| | hydroxypropylmethyl- | 72 |
| | cellulose | |
| | propyleneglycol | 8 |
| covering layer | ethylcellulose (type N14) | 49.1 |
| | sodium laurylsulphate | 2.4 |
| | cetylalcohol | 3.0 |
| | dibutylphthalate | 45.5 |

The ingredients are dissolved in a suitable solvent, for example, isopropanol and/or water, and poured onto a suitable non-stick substrate to form a film with the desired layer thickness and left to dry. The hydrocolloid layer and the covering layer may be prepared separately and stuck together using the binder solution, or the layers may be poured directly onto one another.

For the Examples mentioned above the hydrocolloid layer was poured so that its weight per unit area after drying was about 0.02 g/cm². The covering layer was about 0.015 g/cm² in Example 5 and 0.06 g/cm² in Example 6. 0.02 g/cm² were used for the binder layer.

The layer thicknesses may vary so that the metering per unit area and the technological properties of the film, for example its adhesive qualities or flexibility, can be optimally adjusted.

### Example 7

| Mucoadhesive tablets (1.76% ambroxol HCl) | [%] |
|---|---|
| ambroxol-HCl | 1.76% |
| hydroxypropylcellulose (L-HPC LH 21 ShinEtsu) | 73.31% |
| polyacrylate (carbopol 940) | 24.44% |
| magnesium stearate | 0.49% |

The ingredients are mixed and compressed in a tabletting machine to produce tablets of the desired shape, preferably flat or slightly convex, up to a thickness of about 0.5 to 2 mm.

### Example 8

| Suppositories (500 mg ambroxol HCl) | [g/suppositories] |
|---|---|
| ambroxol-HCl | 0.5 |
| hard fat | 3.0 |

The hard fat is melted in a water bath.

Ambroxol-HCl is suspended in the molten base, poured into a suitable mould, and left to cool until the suppositories harden.

### Example 9

| Mucoadhesive hydrophobic | |
|---|---|
| ointment 30% ambroxol HCl | [%] |
| ambroxol HCl | 30 |
| paraffin | 33.25 |
| polyethylene | 2.5 |
| gelatine | 16.7 |
| pectin | 16.7 |
| sodium carboxymethylcellulose | 16.7 |

### Example 10

| Mucoadhesive hydrophobic | |
|---|---|
| ointment 30% ambroxol HCl | [%] |
| ambroxol HCl | 30.0 |
| polymethyvinylether-co-maleic | 10.0 |
| anhydride | |
| carboxymethylcellulose | 10.0 |
| tragacanth powder | 10.0 |
| paraffin | 38.0 |
| polyethylene | 2.0 |

The hydrocolloids are mixed and placed in the gel consisting of the polyethylene and paraffin fractions. Ambroxol hydrochloride is suspended in this base.

### Example 11

| Hydrophilic ointment (10% ambroxol HCl) | [g] |
|---|---|
| ambroxol-HCl | 10.0 |
| white Vaseline | 31.5 |
| liquid paraffin | 31.5 |
| cetostearylalcohol | 24.3 |
| sodium cetostearylsulphate | 2.7 |

White Vaseline, liquid paraffin, cetostearylalcohol and sodium cetostearylsulphate are melted in the water bath. Ambroxol HCl is suspended therein and the mixture is stirred until cool.

### Example 12

| Hydrophilic O/W cream | | |
|---|---|---|
| (2.1% ambroxol HCl) | | [g] |
| hydrophilic phase | ambroxol-HCl | 2.1 |
| | purified water | 67.9 |
| lipophilic phase | white Vaseline | 10.5 |
| | liquid paraffin | 10.5 |
| | cetostearylalcohol | 8.1 |
| | sodium cetostearylsulphate | 0.9 |

White Vaseline, liquid paraffin, cetostearylalcohol and sodium cetostearylsulphate are melted in the water bath. Ambroxol HCl is dissolved in the heated water, added to the mixture, and stirred until cool.

### Example 13

| Hydrophilic O/W cream | | |
|---|---|---|
| (1.2% ambroxol HCl) | | [g] |
| hydrophilic phase | ambroxol-HCl | 1.2 |
| | purified water | 39.5 |
| | propyleneglycol | 9.9 |
| | polyethyleneglycol-100- | 6.9 |
| | glycerolmonostearate | |
| lipophilic phase | white Vaseline | 25.2 |
| | medium-chain triglyceride | 7.4 |
| | cetylalcohol | 5.95 |
| | glycerol monostearate | 3.95 |

White Vaseline, medium-chain triglyceride, cetylalcohol and glycerol monostearate are melted in the water bath. Purified water, propyleneglycol, and polyethyleneglycol-100-glycerolmonostearate are mixed and heated to about the temperature of the oily phase. Ambroxol-HCl is dissolved in the aqueous mixture. The hydrophilic phase is then added to the lipophilic phase. The mixture is stirred until cool.

### Example 14

| Sodium stearate stick (2.95% ambroxol HCl) | [g] |
|---|---|
| ambroxol-HCl | 3.0 |
| ethanol 96% | 86.0 |
| stearic acid | 6.0 |
| glycerol | 6.0 |
| sodium hydroxide | 0.84 |

Stearic acid, glycerol, and sodium hydroxide are dissolved in ethanol (96%). Ambroxol-HCl is added and the solution is poured into a suitable mould.

### Example 15

| Polyethyleneglycol stick 30% ambroxol HCl | [g] |
|---|---|
| ambroxol-HCl | 15.0 |
| polyethyleneglycol 1000 | 30.0 |
| polyethyleneglycol 600 | 5.0 |

Polyethyleneglycol 1000 and polyethyleneglycol 600 are melted in the water bath, ambroxol-HCl is suspended therein, and the solution is poured into a suitable mould.

1. A topical pharmaceutical composition with anti-inflammatory and local anaesthetic properties comprising ambroxol or a pharmacologically acceptable salt thereof adapted for direct application or administration to the skin or mucosa.

2. The topical pharmaceutical composition according to claim 1, wherein ambroxol is present in the form of its hydrochloride.

3. The topical pharmaceutical composition according to claim 1 in the form of a gel, hydrophilic paste, lotion, or solution.

4. The topical pharmaceutical composition according to claim 3, characterized in that the concentration of ambroxol is from 0.1% to 20%.

5. The topical pharmaceutical composition according to claim 3, characterized in that the concentration of ambroxol is from 0.5% to 5%.

6. The topical composition according to claim 1, in the form of an emulsion, characterised in that the concentration of ambroxol is from 0.1% to 50% (w/w) based on the total mass of the dried hydrophilic layer.

7. The topical composition according to claim 6, wherein the emulsion is an aqueous system, and characterised in that the concentration of ambroxol is from 1.5% to 5%.

8. The topical composition according to claim 6, wherein the emulsion is an anhydrous system, and characterised in that the concentration of ambroxol is from 5% to 30%.

9. The topical pharmaceutical composition according to claim 1 in the form of a suppository, hydrophobic paste, ointment, cream, or stick.

10. The topical composition according to claim 9, in the form of a stick, characterised in that the concentration of ambroxol is from 0.1% to 50% (w/w).

11. The topical composition according to claim 10, characterised in that the concentration of ambroxol is from 2% to 40% (w/w) based on the total mass of the dried hydrophilic layer.

12. The topical pharmaceutical composition according to claim 1 in the form of a mucoadhesive plaster, buccal strip, or mucoadhesive tablet.

13. The topical composition according to claim 12, in the form of a mucoadhesive plaster comprising a dried hydrophilic layer, characterised in that the concentration of ambroxol is from 1% to 50% (w/w) based on the total mass of the dried hydrophilic layer.

14. The topical composition according to claim 13, characterised in that the concentration of ambroxol is from 10% to 30% (w/w) based on the total mass of the dried hydrophilic carrier layer.

15. The topical composition according to claim 12, in the form of a mucoadhesive tablet, characterised in that the concentration of ambroxol is from 0.1% to 30% (w/w).

16. The topical composition according to claim 15, characterised in that the concentration of ambroxol is from 1% to 20% (w/w).

17. The topical composition according to claim 1, wherein the retention time of the ambroxol or one of the pharmaceutically acceptable salts thereof on the skin or mucosa is extended compared to a non-ionic hydrophilic cream containing 0.1% ambroxol according to the 2003 edition of the German Pharmacopoeia.

18. A method for the topical treatment of pain, burning, or itching of the skin or mucosa, comprising the step of applying a composition according to claim 1 to the skin or mucosa of a patient.

19. A method for the treatment of inflammation of the skin or mucosa, comprising the step of applying a composition according to claim 1 to the skin or mucosa of a patient.

20. A method for the treatment of painful or itchy inflammation in the mouth, burning or itching inflammation in the vaginal area, mosquito bites, rashes of allergic, immunological, or idiopathic origin, or itching or burning hemorrhoids, the method comprising the step of applying a composition according to claim 1 to the skin or mucosa of a patient.
